(19) United States
(12) Patent Application Publication
(10) Pub. No.: US 2017/0332622 A1
(43) Pub. Date: Nov. 23, 2017

(54) COMPOSITIONS AND METHODS FOR PRESERVATION AND STORAGE OF BIOLOGICAL MATERIALS

(71) Applicant: The University of North Carolina at Charlotte, Charlotte, NC (US)

(72) Inventor: Gloria D. ELLIOTT, Charlotte, NC (US)

(21) Appl. No.: 15/600,445

(22) Filed: May 19, 2017

Related U.S. Application Data
(60) Provisional application No. 62/338,770, filed on May 19, 2016.

(51) Int. Cl. A01N 1/02; C12N 1/04

(52) U.S. Cl. CPC A01N 1/0221; A01N 1/0284; C12N 1/04

(57) ABSTRACT

In one aspect, solvent systems are described herein. In some embodiments, such a solvent system comprises a disaccharide component, choline halide or choline acetate, and water. The water is present in the solvent system in an amount of 5 weight percent to less than 25 weight percent, and the solvent system is free of crystals. In another aspect, biomolecular compositions are described herein. Such a composition can comprise one or more biological molecules dissolved in a solvent system described herein. In yet another aspect, methods of storing a biological composition are described herein. In some instances, such a method comprises contacting the biological composition with a liquid preservative composition, wherein the liquid preservative composition comprises a solvent system or biomolecular composition described herein.

### CROSS REFERENCE TO RELATED APPLICATIONS

This application claims priority pursuant to 35 U.S.C. §119(e) to U.S. Provisional Patent Application Ser. No. 62/338,770, filed on May 19, 2016, which is hereby incorporated by reference in its entirety.

### FIELD

This invention relates to the preservation and storage of biological materials and, in particular, to compositions and methods for the preservation and storage of biomolecules, cells, tissues and organs.

### BACKGROUND

Over the past decade, vitrification has been widely used for living biological materials as a potential alternative to conventional slow-rate freezing. In vitrification, ice formation can be completely prevented due to rapid cooling rates and/or high cryoprotectant concentrations. However, the rapid cooling rates often required by vitrification can result in non-uniform temperature distributions in the specimen, which leads to non-uniform thermal expansion. Such non-uniform thermal expansion may in turn result in permanent structural damage followed by fracture formation. Additionally, high cryoprotectant concentrations can offer significant challenges, including for larger biological materials such as larger tissues. For instance, larger tissues generally require longer incubation times to enable sufficient concentrations of cryoprotectant agents (CPAs) to be established throughout the entire tissue, which can result in chemical toxicity in the outermost tissues layers, especially when high concentrations of CPAs are used. Another limitation of current vitrification solutions is the need to maintain samples within a small range below T_{g} values to avoid cracking of samples. These temperatures (about −130° C.), however, are difficult to achieve with traditional cryogen-based banking approaches.

Therefore, development of new compositions and methods for preserving biological materials is needed.

### SUMMARY

Compositions and methods are described herein which, in some embodiments, provide one or more advantages compared to previous compositions and methods, including for the preservation and/or storage of biological materials or compositions (including organs and other large tissues). For example, in some cases, compositions and methods described herein provide low critical cooling rates and/or critical heating rates, non-toxicity, and/or higher T_{g} values that enable storage of biological materials at practical temperatures (such as temperatures of −80° C. or above). Compositions and methods described herein, in some instances, can thus significantly simplify the biobanking of vitrified samples. Moreover, in some embodiments, compositions and methods described herein can be used for other biological applications and/or for green chemistry. For example, in some cases, a composition described herein can be a green solvent for biotechnology processes, a reaction medium for water insoluble component, and/or a stabilization medium for enzymes.

In one aspect, solvent systems are described herein. In some embodiments, such a solvent system comprises a disaccharide component, a choline-containing species such as choline halide or choline acetate, and water. Moreover, the water is present in the solvent system in an amount of 5 weight percent to less than 25 weight percent, and the solvent system is free of crystals. Such crystals that are absent from the solvent system can be any crystal or crystalline form of any component of the solvent system or any combination of components of the solvent system. Further, in some cases, the disaccharide component of a solvent system described herein comprises one or more species formed of two glucose units, such as trehalose or trehalose derivative. Additionally, in some instances, a choline halide rather than choline acetate is used, and the choline halide is choline chloride. Moreover, in some embodiments, a molar ratio of the disaccharide component to choline halide or choline acetate ranges from 1:2 to 1:6. Further, in some cases, a solvent system described herein has a glass transition temperature in a range of −40° C. to −120° C. or −50° C. to −60° C. Additionally, in some embodiments, a temperature dependence of viscosity of the solvent system displays Arrhenius behavior.

In another aspect, biomolecular compositions are described herein. Such a composition, in some cases, comprises one or more biological molecules dissolved in a solvent system described herein. For instance, in some embodiments, the solvent system comprises a disaccharide component, choline halide or choline acetate, and water, wherein the water is present in the solvent system in an amount of 5 weight percent to less than 25 weight percent, and the solvent system is free of crystals. Further, the one or more biological molecules can be selected from the group consisting of proteins, enzymes and nucleic acids.

In yet another aspect, methods of storing a biological composition are described herein. In some instances, such a method comprises contacting the biological composition with a liquid preservative composition, wherein the liquid preservative composition comprises a solvent system described herein. For example, in some cases, the liquid preservative composition includes a disaccharide component, choline halide or choline acetate, and water, wherein the water is present in the liquid preservative composition in an amount of 5 weight percent to less than 25 weight percent. Methods described herein, in some embodiments, further comprise converting the liquid preservative composition to an amorphous solid via cooling. Moreover, in some cases, the liquid preservative composition is cooled at a minimum critical cooling rate of 1.5° C./min. The liquid preservative composition may also be cooled and maintained at a temperature of −60° C. to −90° C. In addition, in some instances, a method described herein further comprises heating the amorphous solid at a critical warming rate of 10° C./min to 20° C./min to return the preservative composition to liquid.

These and other embodiments are described in greater detail in the detailed description which follows.

### BRIEF DESCRIPTION OF THE DRAWINGS

FIG. 1 is a graphical illustration of the absorbance spectra values of a protein according to some embodiments of the instant disclosure.

FIG. 2 is a graphical illustration of the absorbance spectra values at 600 nm for the protein in a solvent system according to some embodiments described herein.

FIG. 3A is a graphical illustration of the unfolding temperature, Tₘ, as a function of the weight percent of solvent compositions according to some embodiments described herein.

FIG. 3B is a table depicting thermal unfolding temperature and enthalpy data for a protein in a solvent system according to some embodiments described herein.

FIGS. 4A-4B illustrate thermal scans for fresh and stored formulations of a protein in a solvent solution according to some embodiments described herein.

FIG. 5 is a graphical illustration of the sorption kinetics of solvent compositions according to some embodiments described herein.

FIG. 6 is a graphical illustration of the moisture sorption isotherm for a solvent system according to some embodiments described herein.

FIG. 7 is a graphical illustration of density data associated with various compositions as a function of water content at 40° C. according to some embodiments described herein.

FIG. 8 is a graphical illustration of density data associated with various TCH compositions having molar ratios from 1:3 to 1:7 as a function of temperature according to some embodiments described herein

FIG. 9 is a graphical illustration of the effect of water on viscosity of various compositions t 40° C. according to some embodiments described herein.

FIG. 10 is an Arrhenius plot for solvent compositions at various water contents according to some embodiments described herein.

FIG. 11 is the activation energy of solvent compositions at various water contents according to some embodiments described herein.

FIGS. 12A-12B are graphical illustrations of fragility parameters as a function of water content according to some embodiments described herein.

FIG. 13 is a graphical illustration of the fragility index (m) of various solvent compositions as a function of water content according to some embodiments described herein.

FIG. 14 is a graphical illustration of heating phase of solvent compositions with a cooling and warming rate of 10° C./min according to some embodiments described herein.

FIG. 15 is a graphical illustration of the effect of cooling rates on ice crystal formation of the solvent compositions according to some embodiments described herein.

### DETAILED DESCRIPTION

Embodiments described herein can be understood more readily by reference to the following detailed description, examples and drawings and their previous and following descriptions. Elements, apparatus and methods described herein, however, are not limited to the specific embodiments presented in the detailed description, examples and drawings. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “from 5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

### I. Solvent Systems

In one aspect, solvent systems are described herein. In some embodiments, a solvent system comprises a disaccharide component, choline halide or choline acetate, and water. Moreover, the water is present in the solvent system in an amount of 5 weight percent to less than 25 weight percent, and the solvent system is free of crystals.

Turning now to specific components, solvent systems described herein comprise a disaccharide component. Any disaccharide component not inconsistent with the objectives of the present disclosure may be used. In some cases, the disaccharide component comprises, consists of, or consists essentially of one or more species formed of two glucose units. For example, in some preferred embodiments, the disaccharide component comprises trehalose or trehalose derivative. A trehalose derivative, in some cases, is a derivative of trehalose in which one or more of the hydroxyl groups of trehalose has been replaced with a protecting group or other group in a manner known in carbohydrate chemistry. In other non-limiting embodiments, a trehalose derivative can exhibit various direct or indirect linkages between the saccharide units. As described further below, the use of trehalose or a trehalose derivative in a composition described herein can provide particularly good performance in solvent and/or biological tissue preservation and storage applications.

The disaccharide component of a solvent system described herein can be present in the solvent system in any amount not inconsistent with the objectives of the present disclosure. In some cases, the amount of disaccharide component is selected with respect to an amount of another component of the system, such as the choline-containing component. For example, in some instances, a molar ratio of the disaccharide component to choline halide (or choline acetate) ranges from 1:2 to 1:6. In some cases, the molar ratio is 1:4.

Solvent systems described herein also include a choline-containing component, such as a choline salt. Any choline-containing component or choline salt not inconsistent with the objectives of the present disclosure may be used. In some particularly preferred embodiment, the choline-containing component comprises, consists of, or consists essentially of a choline halide such as choline chloride. In other instances, choline acetate is used. In still other cases, the choline-containing component comprises choline phosphate. Other choline salts may also be used.

The choline-containing species of a solvent system described herein can be present in the solvent system in any amount not inconsistent with the objectives of the present disclosure. In some embodiments, for example, the amount of choline-containing species is selected with respect to an amount of another component of the system, such as the disaccharide component. In some cases, a molar ratio of the disaccharide component to the choline-containing species ranges from 1:2 to 1:6. In some cases, the molar ratio is 1:4.

Solvent systems described herein also include water, particularly in an amount of 5 weight percent to less than 25 weight percent, based on the total weight of the solvent system, or based on the total weight of the disaccharide component, choline-containing component, and water component. In some instances, the water is present in the solvent system in an amount of 10 weight percent to less than 25 weight percent, 12 weight percent to less than 25 weight percent, 15 weight percent to less than 25 weight percent, or 20 weight percent to less than 25 weight percent. In some cases, the water is present in the solvent system in an amount of 5-23, 5-20, 10-23, 10-20, 10-15, 12-23, 12-20, 12-15, 15-23, or 15-20 weight percent, based on the total weight of the solvent system, or based on the sum of the weights of the disaccharide component, choline-containing component, and water component.

As described further hereinbelow, the use of components and amounts described herein can permit a solvent system described herein to exhibit one or more desirable properties, including for solvent applications and/or biological material storage applications. For example, in some cases, a solvent system described herein is free of crystals. Such crystals that are absent from the solvent system can be any crystal or crystalline form of any component of the solvent system or any combination of components of the solvent system.

Further, in some cases, a solvent system described herein has a glass transition temperature in a range of −40° C. to −120° C. For example, in some cases, a solvent system described herein has a glass transition temperature in a range of −40° C. to −100° C., −40° C. to −80° C., −50° C. to −120° C., −50° C. to −100° C., −50° C. to −80° C., −50° C. to −60° C., −60° C. to −120° C., −60° C. to −100° C., or −60° C. to −80° C.

Additionally, in some embodiments, a temperature dependence of viscosity of the solvent system displays Arrhenius behavior, such as in the range of 5-40° C.

Moreover, use of a solvent system described herein, in some cases, can prevent ice formation during cooling of the solvent system and/or cooling of the solvent system plus a biological material containing the solvent system or disposed in the solvent system. For instance, in some cases, less than 10%, less than 5%, or less than 3% of water present in the system (by moles or by mass) forms ice crystals upon cooling or solidification of the solvent system, including at a rate or in a manner described further hereinbelow.

### II. Biomolecular Compositions

In another aspect, biomolecular compositions are described herein. Such a composition, in some cases, comprises one or more biological molecules dissolved in a solvent system described herein. Any solvent system described hereinabove in Section I may be used. For instance, in some embodiments, the solvent system comprises a disaccharide component, choline halide or choline acetate, and water, wherein the water is present in the solvent system in an amount of 5 weight percent to less than 25 weight percent, and the solvent system is free of crystals. Further, the one or more biological molecules can be selected from the group consisting of proteins, enzymes and nucleic acids. Any proteins, enzymes and nucleic acids not inconsistent with the objectives of the present invention may be used, and the identity of the proteins, enzymes and nucleic acid is not particularly limited. However, it is to be understood that, in some cases, the biological molecules are not water soluble or are only minimally soluble in water.

### III. Methods of Storing a Biological Composition

In yet another aspect, methods of storing a biological composition are described herein. In some instances, such a method comprises contacting the biological composition with a liquid preservative composition, wherein the liquid preservative composition comprises a solvent system or biomolecular composition described herein. Any solvent system described hereinabove in Section I or any biomolecular composition described hereinabove in Section II may be used. For example, in some cases, the liquid preservative composition includes a disaccharide component, choline halide or choline acetate, and water, wherein the water is present in the liquid preservative composition in an amount of 5 weight percent to less than 25 weight percent.

Methods described herein, in some embodiments, further comprise converting the liquid preservative composition to an amorphous solid via cooling. Such a cooling step can be carried out in any manner not inconsistent with the objectives of the present disclosure. For example, in some cases, the liquid preservative composition is cooled at a minimum critical cooling rate of 1.5° C./min. The liquid preservative composition may also be cooled and maintained at a temperature of −60° C. to −90° C.

In addition, in some instances, a method described herein further comprises heating the amorphous solid to return the preservative composition to a liquid state. Such a heating step can be carried out in any manner not inconsistent with the objectives of the present disclosure. Moreover, such a heating step can be used to warm, thaw, or devitrify the biological composition following storing for a period of time, such as a period of time of up to 6 months, up to 1 year, up to 5 years, up to 10 years, or up to 20 years. Moreover, any critical warming rate not inconsistent with the objectives of the present disclosure can be used. In some cases, the heating is carried out at a critical warming rate of 10° C./min to 20° C./min.

Similarly, the biological composition can comprise any biological material not inconsistent with the objectives of the present disclosure. In some cases, the biological composition comprises one or more of biological molecules described above, viruses (including bacteriophages), bacteria, cells, engineered cells, tissues, engineered tissues, organs, micro-tissues, and microphysiological systems. In some embodiments, the biological material is a large tissue sample, such as a complete organ.

Additional embodiments will now be further described with reference to the following non-limiting examples.

### Example 1

### Preparation of Trehalose Choline Chloride (TCH) Compositions

High-purity α,α-trehalose dihydrate (i.e., disaccharide component) was purchased from Ferro Pfanstiehl Laboratories (located in Waukegan, Ill.). Choline chloride (i.e., chlorine halide component) was purchased from Sigma-Aldrich (located in St. Louis, Mo.), and deionized water (18 MΩ·cm) was used for exemplary compositions herein that are employed as solvent systems. Solvent compositions of trehalose and choline chloride were prepared by a heating method, in which trehalose and choline chloride (TCH) were combined in a 1:4 molar ratio in a glass jar with a stirring bar and a cap and then heated in a water bath at 80° C. with agitation till a clear liquid was formed (about 3-4 hours).

Ten droplets of water were added to form a completely clear liquid. The solution was diluted up to the desired water content by adding water drop-wise for further uses. Trehalose as well as choline chloride solutions with different concentrations were prepared as control groups. Water content of all samples was measured using a Karl Fischer Titrator. All samples were kept in well-sealed vials after preparation in a desiccator at room temperature. Properties associated with a TCH solvent are in the table below.

TABLE 1
Properties of Example TCH Composition
The Trehalose:Choline Chloride:Water ratio of 1:4:3, 6.5M

| | | |
|---|---|---|
| Glass Transition Temperature | −56.6° | C. |
| Critical Cooling Rate | >1.5° | C./min |
| Critical Warming Rate | 10° | C./min |
| Viscosity at 25° C. | 2.02 | Pa · s |
| Viscosity at 37° C. | 0.96 | Pa · s |

### Example 2

### Solubility of Protein in TCH Solvents

Initial solubility studies were performed on the model protein lysozyme in water. Samples having known concentrations were prepared by weighing the lysozyme and adding the appropriate amount of water to get the desired concentration in mg/mL. The samples were gently inverted several times and stored at room temperature for 1 hour to overnight to allow the protein to rehydrate. Solubility limits were determined via turbidimetric assay.

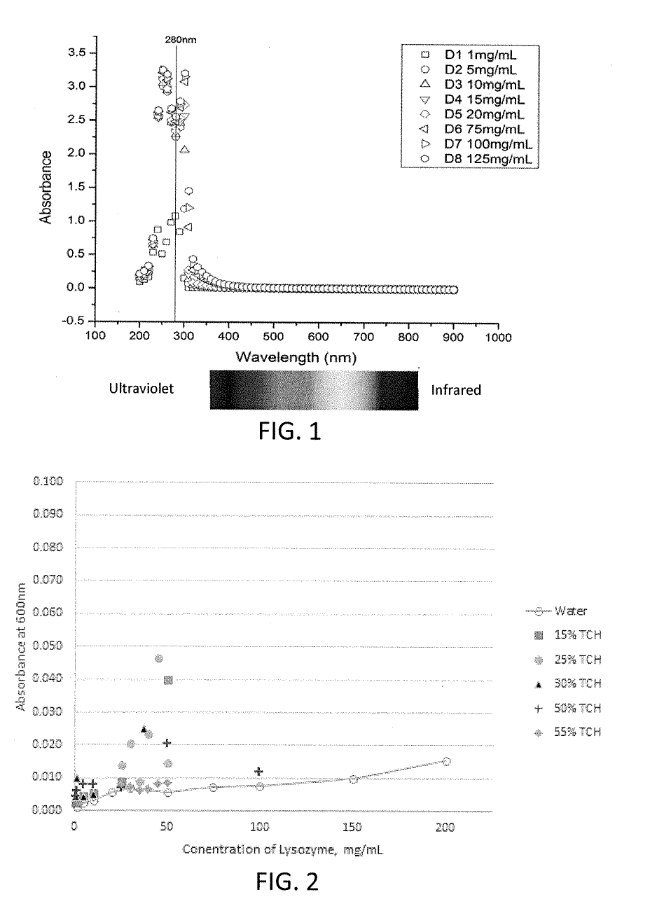

To determine the solubility limits, aliquots of 150 μL were taken from each sample and added to a 96 half-well plate. The samples were then placed in a Biotek Synergy plate reader and absorbance was measured from 200 nm to 900 nm. As FIG. 1 illustrates, lysozyme has a characteristic fluorescence at 280 nm that can be used along with Beer-Lambert's law to measure the concentration, however, for samples above 5 mg/mL, the instrument sensor becomes saturated and the readings are not useful for determining concentration. Therefore, to determine solubility limits, the absorbance at 600 nm was used to indicate the presence of aggregate or particulate in suspension. At this wavelength, far from the characteristic absorbance, any increase in absorbance is an indicator that the protein is not completely in solution. A baseline was established by measuring the absorbance of pure water or TCH solution without any lysozyme. Samples were measured in triplicate and the averages are plotted in the accompanying figures.

To determine the effect of TCH on the solubility of lysozyme, values for the solubility of lysozyme in TCH formulations as a function of water content were obtained. FIG. 1 shows the absorbance spectra of lysozyme at concentrations from 1 mg/ml to 125 mg/ml. There is a characteristic peak proximate 280 nm that can be used can be used along with Beer-Lambert's law to determine concentrations, however, the spectra is near zero away from this peak. For this reason, the absorbance at 600 nm was used to indicate solubility challenges since an increase in absorbance at 600 nm would suggest aggregation or settling of the protein.

As indicated by the line plotted in FIG. 2, in pure water the lysozyme showed no indication of solubility issues up to the maximum concentration tested, 200 mg/ml. Compared to FIG. 1 with peaks near 3, the highest absorbance measured in FIG. 2 is 0.05. While the increase in absorbance is noticeable at this scale, it is, for practical purposes zero when considering the scale of FIG. 2.

As FIG. 2 further illustrates, the absorbance values are slightly higher in intermediate solutions of 15% and 25% TCH with small absorption increases observed at around 40 mg/ml. For each sample measured, the absorbance at 600 nm was generally low, falling below 0.05, suggesting that the protein is soluble in these compositions.

At concentrations below 40 mg/mL absorbance values were comparable to those of lysozyme in pure water. The solution with the highest concentration of TCH (i.e., TCH at 55%), had some of the lowest absorbance values suggesting that solubility of lysozyme may be better in these solutions. The results indicate that the model protein lysozyme is readily soluble in TCH solutions.

### Example 3

### Protein Stabilization Via TCH Solvents

TCH solutions were prepared, water was added, and the water content was measured using Karl Fischer titration until the desired solutions were achieved. Lysozyme was weighed into the container and the appropriate amount of TCH was added to achieve a solution with a concentration of approximately 1 mg/mL. Samples were gently inverted several times and left at room temperature for one hour or overnight to allow the lysozyme to rehydrate and go into solution. Some samples were dried by evaporative drying and rehydrated prior to scanning Another sample of lysozyme in 0.1M sodium acetate buffer pH 4 was prepared for comparison.

To determine the stability of lysozyme in TCH over time, a set of samples was prepared at stable moisture contents and stored at the appropriate humidity at room temperature for a period of five weeks. These samples were prepared with a protein concentration of 10 mg/mL prior to storage. After five weeks, they were diluted to approximately 1 mg/mL and scanned using differential scanning calorimetry (DSC).

A Micro-cal VP DSC was used for determining the thermal stability of the protein. A baseline was established by scanning the TCH solution without protein at least three times until repeatability was evident. The TCH solutions used for the baseline scans are identical to the sample solutions, without protein. The baseline scans of the solutions without protein help eliminate noise resultant from variation in the sample cells. Samples with protein concentrations of approximately 1 mg/mL were added to the sample cell and scanned several times. Cells were rinsed with water and TCH solution between samples. The concentration of lysozyme in each sample was verified prior to scanning by reading the absorbance at 280 nm on the Biotek Synergy plate reader. The exact concentrations were used to normalize enthalpy data.

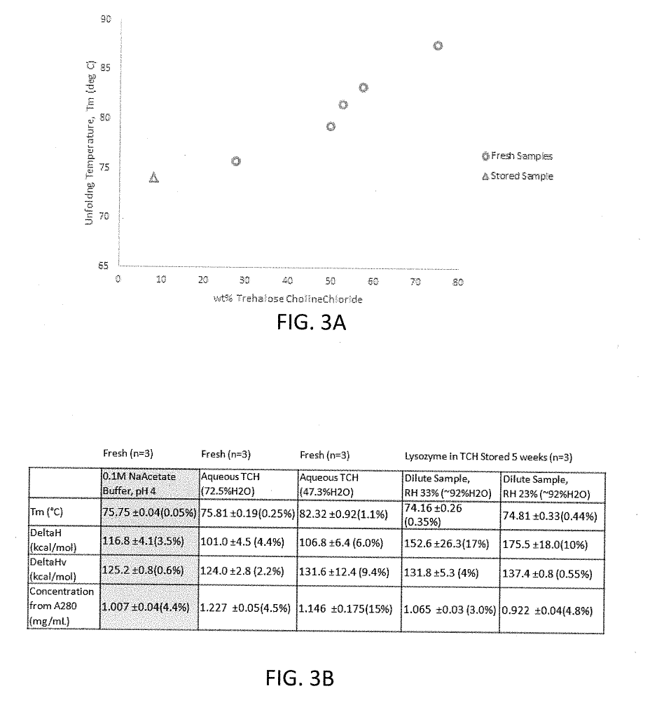

The stabilization effects of TCH on the model protein lysozyme were performed to determine how the concentration of TCH affects the thermal unfolding temperature of the protein. The results of the protein stabilization study shown in FIG. 3A are presented as a plot of the thermal unfolding temperature of lysozyme versus the weight percent of TCH in solution. In FIG. 3, the “fresh” samples were for freshly prepared TCH samples of varying content, and the “stored” samples were prepared, stored for five weeks, and diluted to appropriate concentrations just prior to scanning. The thermal unfolding temperature of lysozyme increases with increasing concentrations of TCH indicating increased thermal stability of lysozyme in the solutions. The stored samples have a slightly higher unfolding temperature which indicates that the thermal stability of the protein has not changed.

The DSC thermal traces were fit in OriginPro with a non-two-state model that includes an output of both calorimetric enthalpy and Van't Hoff enthalpy, Hᵥ. The calorimetric enthalpy is determined by the area under the curve and depends on the selected baseline as well as the normalized concentration. The Van't Hoff enthalpy depends on the shape of the curve and, for proteins, the number of steps in the unfolding process. The number of intermediate steps in the unfolding process can be determined by taking the ratio of calorimetric enthalpy to Van't Hoff enthalpy. For a truly two-state process, the calorimetric and Van't Hoff enthalpy ratio would be 1; and the values would be the same. If the values are unequal, it may indicate intermediate unfolding steps, a non-two state process, or it could be the result of a normalized concentration that does not capture exactly the amount of correctly folded protein. The data is shown in the table depicted in FIG. 3B for freshly prepared and stored samples with varying TCH content. Each concentration was prepared and scanned in triplicate (n=3). The enthalpies obtained from DSC are in FIG. 3B.

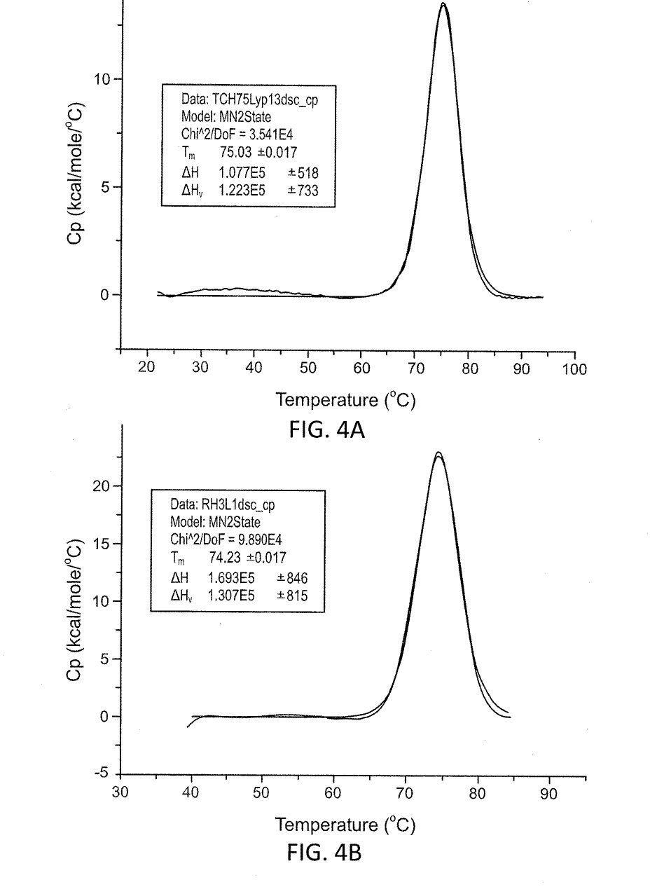

Lysozyme typically has a two-step unfolding process and the smooth DSC endotherms do not suggest any intermediate steps, see FIGS. 4A-4B. The calorimetric enthalpies for lysozyme in a sodium acetate buffer range from 103 to 105 kcal/mol². Lysozyme in NaH₂PO₄ had slightly higher enthalpy values, from 122 to 129 kcal/mol², with the ratio of calorimetric to Van't Hoff enthalpies close to 1.

### Example 4

### Sorption Characteristics of TCH Solvents

TCH solutions were prepared from the materials noted above. The TCH mixture was prepared gravimetrically in a glass vial. The choline chloride was weighed directly into the vial while the trehalose was weighed on paper and added to the vial. A 1:4 molar ratio of trehalose dihydrate and choline chloride was added to the vial with ten droplets of water. The sample was heated in a 75-80° C. water bath with a stir bar until it became a clear, homogenous liquid. If the sample did not become clear within a few hours, 5-10 additional drops of water were added. The sample solution was stored in the closed vial at room temperature until use.

For the moisture sorption studies, small samples of the viscous TCH sample were pipetted onto petri dishes of known weight and dried by evaporative drying for 24-30 hours. A glass coverslip was tared on the scale and 5-10% of the dried sample was transferred onto the coverslip. The coverslip with sample was added to a volumetric Karl Fisher titrator to determine the mass of water present and enable reporting of the percentage of water by weight. The remaining sample was weighed and placed in a relative humidity jar. Assuming a homogenous sample, the dry weight (anhydrous) was determined by subtracting the measured water content from the initial analyzed mass. Samples ranged from 500 mg to 822 mg dry weight, with most falling between 500 mg and 700 mg. Every few days, samples were briefly removed from the jars and weighed to determine the amount of water gained or lost. Samples were measured until crystals formed or until the mass equilibrated.

Dry weight determinations were verified after samples equilibrated by bake-out in a gravity convection oven (available from VWR, West Chester, Pa.) at 95° C. for 48 hours. Samples were then moved to a desiccator with phosphorus pentoxide and weighted inside a low humidity chamber until there was no change in weight, approximately one week. The dry weight measured by bake-out was used for calculating moisture content of the samples.

The moisture sorption isotherm was plotted and fit to the standard BET model in OriginPro 8. Water content is expressed as either g H₂O/g dry weight or as weight percent water (g H₂O/mass sample).

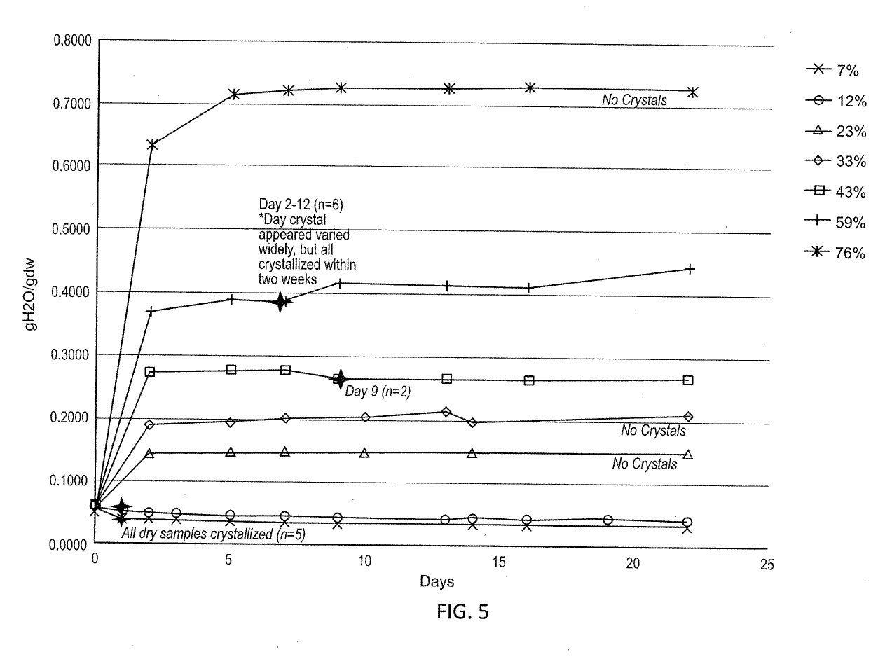

Moisture sorption characteristics of the TCH composition as a function of water content can have a significant effect on the stability of the solution. Samples were observed for crystal formation throughout these experiments. FIG. 5 shows the moisture sorption kinetics for the TCH 1:4 molar ratios at room temperature for all of the relative humidity levels tested. The first day crystals were observed is marked with a star and “n” is the number of samples that crystallized on each day.

Samples picked up water quickly in the first few days and then gradually reached equilibrium within two to three weeks. Prior to storage, the dried samples with 0.02-0.08 grams of water per gram dry weight appeared cloudy and had a needle-like crystal structure confirmed via optical microscopy. At relative humidity (RH) values below 23%, the samples continued to lose moisture and remained cloudy.

At relative humidity values of 23% RH and higher, all samples became clear within the first day or two and remained clear throughout the duration of this study. At 43% RH and 59% RH, the components started to precipitate out of solution. Samples at 75.5% RH appeared water-like and only one of five replicates had components precipitate out of solution. The lack of crystals at the highest RH tested indicates that the solutions took on moisture quickly enough to bypass the supersaturated region and fully solubilize the sample.

As the dried samples were exposed to higher relative humidity values and began to take on moisture, the needle-like crystals dissolved and the solution appeared amorphous. Samples became reflective and appeared wet. A dried sample that was sitting at room temperature exposed to the relative humidity of the environment for less than ten minutes had a barely visible crystal structure.

Compared to the moisture sorption isotherms of anhydrous trehalose, the TCH composition absorbs water much more quickly. The well-known Brunauer Emmett Teller (BET) was used to generate a model isotherm. The BET equation is given as:

$$BET \text{:} W ( {a}_{0} ) = \frac{{W}_{B} {C}_{B} {a}_{0}}{( 1 - {a}_{0} ) ( 1 + ( {C}_{B} - 1 ) {a}_{0} )} \qquad ( Eq . 1 )$$

where W(a₀) is the moisture content of the sample at water activity a₀, W_{B} is the monolayer value (a representation of the number of hydrophilic binding sites for water) and C_{B} is an energy constant. The free energy constant, C_{B}, is related to the difference of free enthalpy or chemical potential of the sorbate molecules in their liquid state and in the monolayer and is represented by:

$${C}_{B} = k exp [ \frac{( Δ {H}_{a} - Δ {G}_{L} )}{RT} ] \qquad ( Eq . 2 )$$

where ΔHₐ is the enthalpy of adsorption, ΔG_{L} is the enthalpy associated with condensation of multilayers as a liquid, and k is a measure of the entropy change associated with adsorption. The results associated with curve fitting the TCH adsorption data and obtaining model parameters for the BET equation from Origin Pro are in the table below, which can be used to calculate moisture content in the BET equation above.

TABLE 2
Model Adsorption Parameters for TCH

| | |
|---|---|
| W_{B} | 0.20237 |
| C_{B} | 3.00417 |
| r² | 0.97809 |

The addition of choline chloride to the composition changed the isotherm from a Type 2 to a Type 3. The Type 3 isotherm, which is characterized by a steady increase in moisture content over the entire humidity range, indicates multilayer absorption without the initial monolayer formation of a Type 2 isotherm. The shift in the isotherm from a Type 2 to Type 3 also suggests that moisture is distributed evenly throughout the bulk of the material instead of accumulating on the surface first. The absorption process for a substance with a Type 3 isotherm is similar to the process of diffusion and correlates with a more stable liquid state. Characteristics associated with the different types of adsorption isotherms are generally known in the art so not described herein.

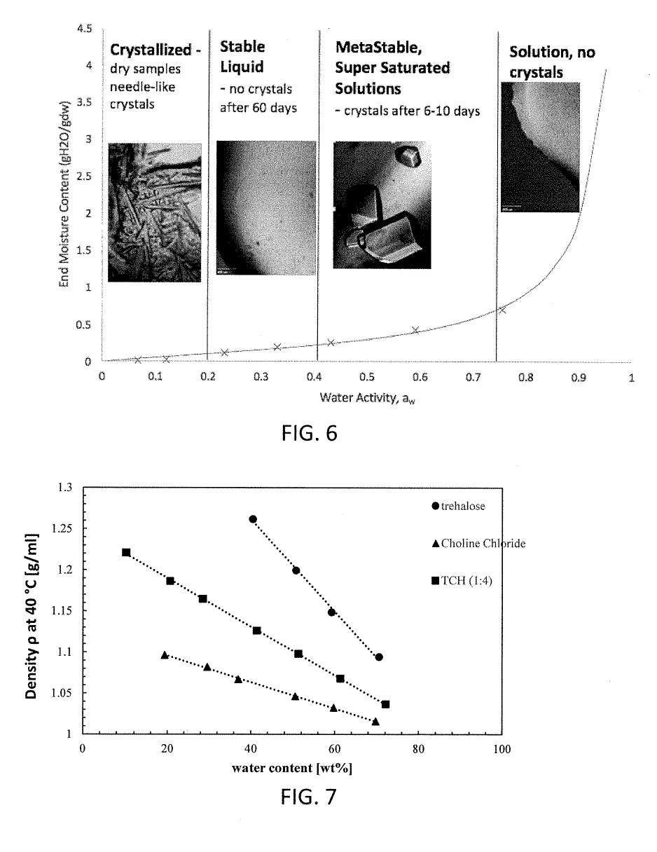

During the sorption experiments, four regions were discerned and approximated in FIG. 6. Dry samples with about 0.03 or less grams of water per gram dry weight appeared cloudy and revealed needle-like crystals as shown previously. There appeared to be a stable region where samples of TCH with water content between about 0.1 and 0.2 grams water per gram dry weight stored at RH of 23 and 33%, respectively, showed no visible signs of crystallization for the duration of this study, even over 60 days. Above this region was a metastable, supersaturated region that became liquid prior to crystals precipitating out of solution. The water content for these samples was around 0.4 grams water per gram dry weight (approximately 30 wt. % water), which is below the solubility limit of pure trehalose, 53% water by weight.

The stable range of water content produced from this study corresponds to between about 9.8 and 16.5% water, by weight with, crystallization observed in samples with less than 5% water or more than 20.6% water. Thus, water should be present in the solvent systems in amounts of 5 wt. % to less than 25 wt. %, and in some aspects, of 5 wt. % to less than 20 wt. %. The crystals could be observed by the naked eye and confirmed with optical microscopy. The crystals have a very similar structure to the crystals formed from a supersaturated solution of pure trehalose. Supersaturated choline chloride forms small crystals and the solution appears cloudy in contrast to the large, distinct trehalose crystals.

Samples stored in humid environments, RH of 75%, hydrated very quickly and did not form crystals. The water content of these solutions varied from about 40 to 42% (grams water per gram solution), which indicates that the trehalose and choline chloride components are fully soluble at the moisture content maintained at this RH. The solubility limit for pure trehalose is reported as ranging from 42.3% (grams trehalose per gram solution) at 10° C. to 59.7% at 40° C. At 20° C., the solubility is reported as 46.6%, which corresponds to a water content of 53.4% (grams water per gram solution).

This data suggests that there is a range of water contents over which the trehalose choline chloride 1:4 composition remains stable without crystallization of components and devoid of crystals. When stored at relative humidity values of 23 to 33% (typical human comfort environmental conditions maintained in indoor environments), the water content of the solution reached an equilibrium of 0.15 and 0.21 grams water per gram dry weight, respectively. Crystallization did occur for samples with water contents below 0.05 and above 0.26 grams water per gram dry weight (i.e., water contents of between 5 and 26 wt. %). Controlling moisture content in the compositions described herein can allow for long term storage and stability of proteins and/or protein formulations.

TCH compositions having a 1:4 molar ratio of trehalose dihydrate and choline chloride appear to be a good solvent for the model protein lysozyme. The protein was soluble in such compositions up to at least 40 mg/mL at all water content levels tested (0.01 to 0.70 grams water per gram dry weight) and showed no sign of degradation after five weeks of storage in stable solutions where the moisture content and/or humidity were controlled. The TCH compositions described herein provide deep eutectic compositions useful for liquid protein formulations.

### Example 5

### Physical Properties

### A. Density

The densities of trehalose, choline chloride and TCH (1:4) diluted with different percentage of water at various temperatures were detected. The different water content of the different samples is in the table below.

TABLE 3
Water content of trehalose, choline chloride,
and TCH with a molar ratio of 1:4.

| | | Choline | |
|---|---|---|---|
| | Trehalose | chloride | Trehalose:Choline |
| | solutions | solutions | Chloride (TCH, 1:4) |
| Water content | 40.46 | 19.33 | 10.17 |
| (% w/w) | 50.7 | 29.623 | 20.65 |
| | 59.25 | 37.091 | 28.51 |
| | 70.545 | 50.543 | 41.4 |
| | | 59.778 | 51.27 |
| | | 69.767 | 61.37 |
| | | | 72.135 |

The behavior of densities depends on temperature and water content. Results at 40° C. in FIG. 7 showed that the density of TCH decreased linearly with the increasing water content following a function y=−0.003x+1.2491, R²=0.9997 (where y=density; x=water weight percentage in TCH). With these formulas, the density of the diluted components can be calculated with known amount of water dilution.

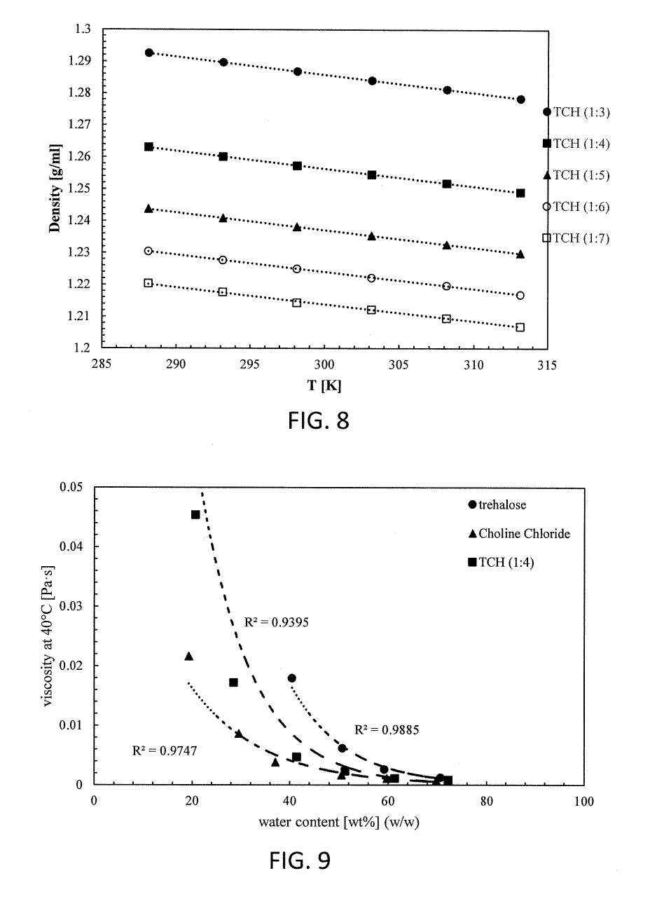

The effect of temperature on densities of TCH at different ratios is depicted in FIG. 8. The increase in temperature results in more molecular activity and mobility, which increases the solution molar volume and eventually reduces density. Estimated densities of TCH at all molar ratios were less than 1.3 g/ml. The reduction in density was linear for all studied TCH. It should be noted that the density of the TCH decreases as the salt molar ratio increases.

### B. Temperature and Water Content Dependent Viscosity

Viscosity data is useful in the design stage of preservation processes and can be used for the selection of optimum ratio of salt and hydrogen bond donor. The viscosities of all samples were fitted using an Arrhenius model per the equation below:

$$η = {η}_{0} {e}^{- \frac{{E}_{a}}{RT}} \qquad ( Eq . 3 )$$

where η is the viscosity, η₀ is a pre-exponential constant, Eₐ is the activation energy, R is the gas constant (8.314 J mol⁻¹ K⁻¹), and T is the temperature in Kelvin. Values of η₀ and Eₐ are shown in the table below.

TABLE 4
Viscosity-temperature model parameters

| | Water content | η₀ | Eₐ | |
|---|---|---|---|---|
| | [wt %] | [Pa · s] | [kJ/mol] | R² |
| Trehalose solutions | 40.46 | 6 × 10⁻⁹ | 38.26 | 0.8998 |
| | 50.7 | 1 × 10⁻⁷ | 27.7 | 0.9947 |
| | 59.25 | 2 × 10⁻⁷ | 23.06 | 0.9976 |
| | 70.545 | 2 × 10⁻⁷ | 25.11 | 0.9994 |
| Choline chloride | 19.33 | 7 × 10⁻⁷ | 26.98 | 0.9979 |
| solutions | 29.623 | 2 × 10⁻⁶ | 21.70 | 0.9964 |
| | 37.091 | 3 × 10⁻⁶ | 18.72 | 0.998 |
| | 50.543 | 2 × 10⁻⁶ | 17.85 | 0.9988 |
| | 59.778 | 1 × 10⁻⁶ | 17.71 | 0.9992 |
| | 69.767 | 6 × 10⁻⁷ | 18.44 | 0.9995 |
| Trehalose-choline | 10.17 | 1 × 10⁻⁸ | 44.16 | 1 |
| chloride solutions | 20.65 | 3 × 10⁻⁸ | 36.48 | 0.9966 |
| | 28.51 | 1 × 10⁻⁷ | 30.75 | 0.9965 |
| | 41.4 | 8 × 10⁻⁷ | 22.45 | 0.9963 |
| | 51.27 | 8 × 10⁻⁷ | 20.63 | 0.9979 |
| | 61.37 | 4 × 10⁻⁷ | 20.72 | 0.9998 |
| | 72.135 | 1 × 10⁻⁶ | 16.85 | 0.9992 |

In general, viscosity is affected by water content and temperature. FIG. 9 displays the water effect on viscosities of trehalose, choline chloride and TCH (1:4) at 40° C. It is seen that the viscosities decreased with increasing moisture. TCH has more sensitivity to water than choline chloride, but less than trehalose, which may be associated with its ability to bind with water by hydrogen bonding. Similar changes of water-dependent viscosity have been found at other temperatures, for example, between 5 and 40° C.

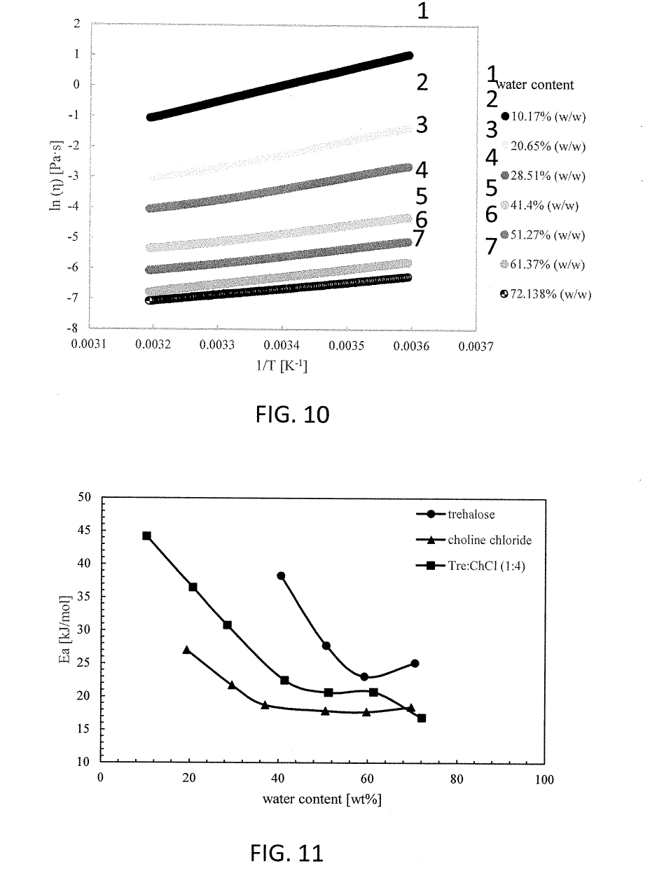

The temperature-dependent viscosity of TCH solutions at 5-40° C. displays Arrhenius behavior as shown in FIG. 10, in which the natural logarithm of viscosity is plotted against inverse temperature. The viscosity of the TCH solutions decreased as the water content and temperature increased. Similar behavior of trehalose and choline chloride solutions was observed. In general, the increase in temperature results in increasing average speed of the molecules in the liquid which decreases the average intermolecular forces and consequently reduces the fluid resistance to flow which is termed as the viscosity.

FIG. 11 shows the activation energy decreasing linearly as the water content increases up to about 41.4% (w/w), indicating that the viscosity is more sensitive to temperature changes at low water contents. The viscosity varies between 22.45 kJ/mol (41.4% (w/w) water content) and 44.16 kJ/mol (10.7% (w/w) water content).

### C. Fragility

Fragility is the deviation of the temperature dependence of viscosity (or α-relaxation) from Arrhenius behavior. It is commonly used to classify the strong-fragile characteristics of glass-former of the glass transition region. A more fragile composition will increase in viscosity more steeply on cooling within the glass transition region compared to a strong composition.

Fragility can be obtained directly from data intermediate viscosities using the F_{1/2} fragility, which describes the degree of departure from an Arrhenius process. F_{1/2} is defined to vary between 0, for a very strong liquid, and 1 for a very fragile hypothetical liquid by the equation

$${F}_{1 / 2} = \frac{2 {T}_{g}}{{T}_{1 / 2}} - 1 \qquad ( Eq . 4 )$$

where T_{1/2} is the temperature at which the viscosity reaches a point half-way between its value at T_{g} (10¹² Pa·s) and η₀ (10⁻⁵ Pa·s) on a logarithmic scale. T_{1/2} therefore occurs at the temperature at which viscosity equals 10³⁵ Pa·s. F_{1/2} was calculated here using a T_{g} value predicted by Eq. 4 with an assumed viscosity of 10¹² Pa·s. The relation between F_{1/2} and the m fragility is as follows:

F_{1/2}=(m−17)/(m+17) (Eq. 5)

where 17 orders of magnitude are assumed to separate T_{g} from the high-temperature extreme for viscosity and 16 would be used for the case of relaxation.

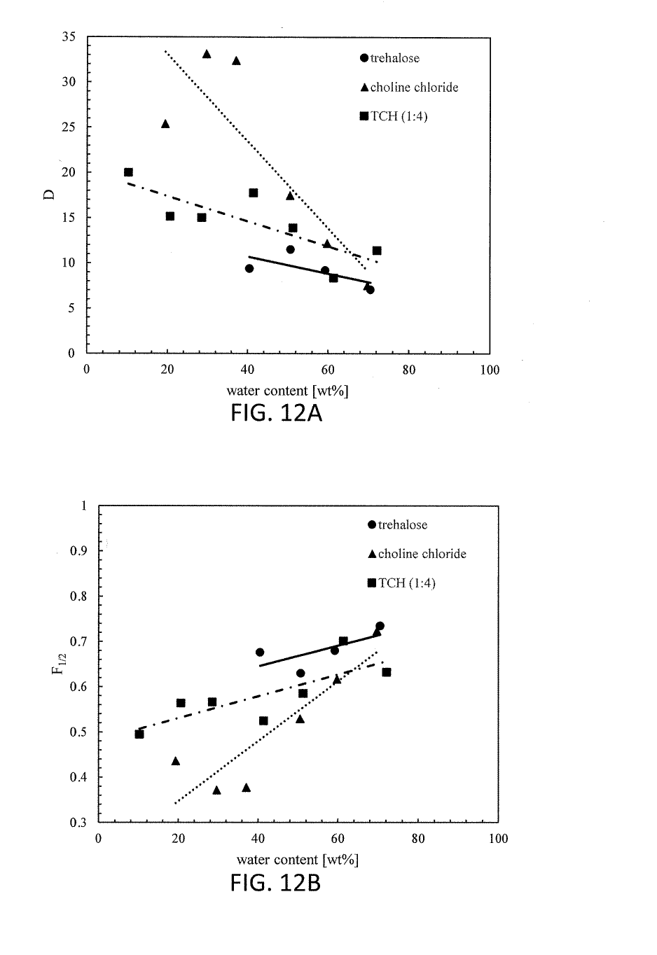

The fragility parameters D and F_{1/2} are given in FIGS. 12A-12B as functions of the water content. Both figures indicate that fragility increases with decreasing D and increasing F_{1/2} values. It was further shown that fragility increased with increasing water content for trehalose, choline chloride and TCH (1:4) and the effect of moisture on fragility of TCH has the similar slope with that of trehalose.

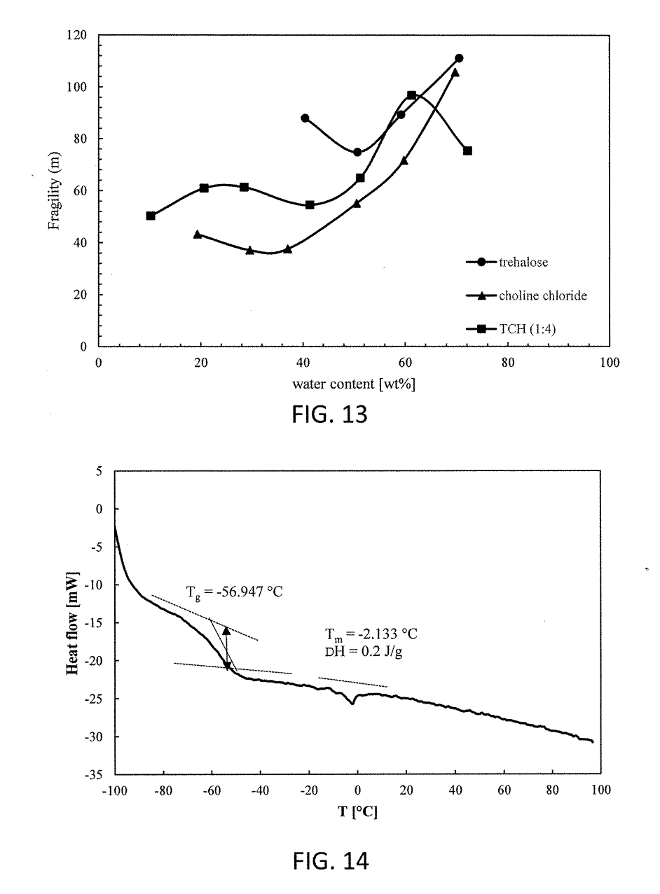

The fragility index (m) profiles calculated by Eq. 5 of trehalose, choline chloride and TCH solutions related to water content are shown in FIG. 13. It was found that the solutions with choline chloride have a lower fragility index as the water content below than 40%. Above that, choline chloride has a strong influence on the fragility of trehalose-choline chloride solutions.

The fragility of trehalose-choline chloride solutions increased significantly and behaved in a manner similar to that of choline chloride solutions, whereas trehalose solutions displays more fragile and has an increase on fragility index as water content above 50% (w/w). The fragility information may be used to select the ratios of salt and/or hydrogen bond donor for solvents described herein.

### D. Ice Crystal Formation and Glass Formation

DSC was used to study glass transition behavior. Upon heating the eutectic mixture having a trehalose:choline chloride:water ratio of 1:4:3 from −100° C. to 100° C., a signature of a glass transition with the midpoint temperature (T_{g}) of −56.947° C. was observed and shown in FIG. 14.

The thermal behavior is consistent with a glassy material that undergoes glass transition but does not crystallize upon heating. An endotherm with the peak temperature of −2.133° C. and the enthalpy of 0.2 J/g followed, which displays a melting phase of co-crystallization. The liquid is viscous (with a predicted value of 2.57 Pa·s) when handled at room temperature (20° C.), which may result in 1.45% of the water forming ice crystal in the eutectic mixture.

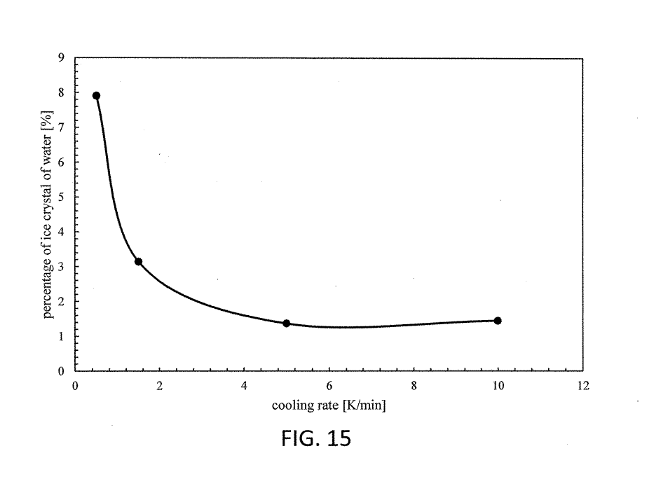

The effect of cooling rates on the amount of ice crystal formation was studied using DSC. Based on the ΔH of DI water as a value of 333.55 J/g, the amount of ice crystal formed by different cooling rates were calculated by dividing the ΔH of each sample by 333.55 J/g and by water content of each sample, shown in FIG. 15 as a function of cooling rate. The ice crystal formed by water significantly decreased as the cooling rate increased and further reached below 2% as the cooling rate achieved above 3° C./min. The eutectic mixture can easily form a glassy state.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A solvent system comprising:
a disaccharide component, choline halide or choline acetate, and water, wherein the water is present in the solvent system in an amount of 5 weight percent to less than 25 weight percent, and the solvent system is free of crystals.

2. The solvent system of claim 1, wherein the disaccharide component comprises one or more species formed of two glucose units.

3. The solvent system of claim 2, wherein the disaccharide component comprises trehalose or trehalose derivative.

4. The solvent system of claim 1, wherein the solvent system comprises choline halide and the choline halide is choline chloride.

5. The solvent system of claim 1, wherein a molar ratio of the disaccharide component to choline halide or choline acetate ranges from 1:2 to 1:6.

6. The solvent system or claim 5, wherein the molar ratio is 1:4.

7. The solvent system of claim 1 having a glass transition temperature in a range of −40° C. to −120° C.

8. The solvent system of claim 1, wherein the water is present in an amount of 15 to 23 weight percent of the solvent system.

9. The solvent system of claim 1, wherein temperature dependence of viscosity of the solvent system displays Arrhenius behavior.

10. A biomolecular composition comprising:
one or more biological molecules dissolved in a solvent system, the solvent system comprising a disaccharide component, choline halide or choline acetate, and water, wherein the water is present in the solvent system in an amount of 5 weight percent to less than 25 weight percent, and the solvent system is free of crystals.

11. The biomolecular composition of claim 10, wherein the one or more biological molecules is selected from the group consisting of proteins, enzymes and nucleic acids.

12. The biomolecular composition of claim 10, wherein the disaccharide component comprises one or more species formed of two glucose units.

13. The biomolecular composition of claim 12, wherein the disaccharide component comprises trehalose or trehalose derivative.

14. The biomolecular composition of claim 10, wherein the solvent system comprises choline halide and the choline halide is choline chloride.

15. The biomolecular composition of claim 10, wherein a molar ratio of the disaccharide component to choline halide or choline acetate ranges from 1:2 to 1:6.

16. The biomolecular composition of claim 15, wherein the molar ratio is 1:4.

17. The biomolecular composition of claim 10 having a glass transition temperature in a range of −40° C. to −120° C.

18. The biomolecular composition of claim 10, wherein the water is present in an amount of 15 to 23 weight percent of the solvent system.

19. A method of storing a biological composition comprising:
contacting the biological composition with a liquid preservative composition, the liquid preservative composition including a disaccharide component, choline halide or choline acetate and water, wherein the water is present in the liquid preservative composition in an amount of 5 weight percent to less than 25 weight percent; and
converting the liquid preservative composition to an amorphous solid via cooling.

20. The method of claim 19, wherein the liquid preservative composition is cooled at a minimum critical cooling rate of 1.5° C./min.

21. The method of claim 19, wherein the disaccharide component comprises one or more species formed of two glucose units.

22. The method of claim 21, wherein the disaccharide component comprises trehalose or trehalose derivative.

23. The method of claim 19, wherein the liquid preservative composition comprises choline halide and the choline halide is choline chloride.

24. The method of claim 19, wherein a molar ratio of disaccharide component to choline halide or choline acetate ranges from 1:2 to 1:6.

25. The method claim 19, wherein the liquid preservative composition is cooled and maintained at a temperature of −60° C. to −90° C.

26. The method of claim 19 further comprising heating the amorphous solid at a critical warming rate of 10° C./min to 20° C./min to return the preservative composition to liquid.

27. The method of claim 19, wherein the biological composition comprises biomolecules, viruses, bacteria, cells, engineered cells, tissues, micro-tissues, organs or microphysiological systems.
